(19) United States
(12) Patent Application Publication
(10) Pub. No.: US 2024/0180796 A1
(43) Pub. Date: Jun. 6, 2024

(54) COMPOSITION FOR TREATING A KERATINOUS SURFACE

(71) Applicant: Givaudan SA, VERNIER (CH)

(72) Inventors: Sergio DI MARTINO, Upper Saddle River, NJ (US); Ralph GENCARELLI, Fairfield, NJ (US); Muhammad MIRZA, Berkeley Heights, NJ (US)

(21) Appl. No.: 18/285,017

(22) PCT Filed: Apr. 20, 2022
(86) PCT No.: PCT/EP2022/060335

§ 371 Date: Sep. 29, 2023

Related U.S. Application Data
(60) Provisional application No. 63/177,938, filed on Apr. 21, 2021.

(51) Int. Cl. A61K 8/11; A61K 8/04; A61K 8/34; A61K 8/73; A61Q 5/02

(52) U.S. Cl. CPC A61K 8/11; A61K 8/046; A61K 8/34; A61K 8/73; A61K 8/732; A61Q 5/02; A61K 2800/31; A61K 2800/56

(57) ABSTRACT

Disclosed is an anhydrous composition for treating a keratinous surface, in particular hair. The composition comprises:
At least one cosmetically acceptable excipient;
At least one perfume ingredient that is entrapped in a water-soluble matrix;
An alcohol;

The amount of the alcohol in the composition is 1-30 wt.-%, preferably 2-20 wt.-%, more preferably 3-10 wt.-%, relative to the total weight of the composition.

The present invention relates to an anhydrous composition for treating a keratinous surface, as well as to a method for treating a keratinous surface.

The practice of regularly washing head hair has become ubiquitous in modern society. Frequently, the hair is washed using a conventional liquid or gel shampoo, which is applied to the hair and then rinsed out with water. However, dry shampoos, which do not require the use of water, are becoming increasingly popular. The use of a dry shampoo can save time and provide added convenience since no rinsing with water is needed; rather, removal of the dry shampoo can be effected simply by brushing or blotting it from the scalp. Furthermore, it has been suggested that excessively frequent wetwashing can be associated with damage to the hair, particularly for those with fine hair. The use of dry shampoos may therefore provide an alternative or complementary method for maintaining the cleanliness and appearance of the hair without incurring the damaging effects of excessive washing in water.

Dry shampoos typically contain perfumes that are expected to deliver longlasting and pleasant odors. However, it is often not possible to achieve this goal if the perfumes are incorporated into such products in neat form, as socalled “free oil”. For this reason, it has been proposed in US 2017/020755 A1 to encapsulate the perfume in starch capsules. While such dry shampoos provide improved olfactive benefit, there is still a need for such products with improved cleaning power.

It is therefore a problem underlying the present invention to overcome the aforementioned shortcomings in the prior art. In particular, it is a problem underlying the present invention to improve the treatment of keratinous surfaces, and more specifically the cleaning of hair with dry shampoos. Furthermore, the styling of the hair after treatment should be improved.

These problems are solved by the subject-matter of the independent claims.

In a first aspect, the present invention relates to an anhydrous composition for treating a keratinous surface, in particular hair. The composition comprises:
At least one cosmetically acceptable excipient;
At least one perfume ingredient that is entrapped in a water-soluble matrix;
An alcohol;

The amount of the alcohol in the composition is 1-30 wt.-%, preferably 2-20 wt.-%, more preferably 3-10 wt.-%, relative to the total weight of the composition.

In the present context, that the “at least one perfume ingredient is entrapped in a water-soluble matrix” is synonymous with the at least one perfume ingredient being encapsulated in the water-soluble matrix material. The matrix material is at least to some degree alcohol-stable.

The alcohol in the composition helps to treat the keratinous surface. When used in a dry shampoo, the alcohol improves the cleaning of the hair from dirt, oil and/or sebum, as it functions as a carrier for absorbent materials contained in the dry shampoo. The alcohol also provides more volume to the hair, as well helps with styling. Moreover, it evaporates quickly after it hits hair roots and absorbs body heat. This creates a cooling effect that soothes an itchy, dirty scalp.

The present invention is based on the surprising finding that the water-soluble matrix entrapping the at least one perfume ingredient is stable in the anhydrous composition, despite the presence of alcohol in the amounts provided herein above.

Perfume ingredients are volatile and often prone to decomposition in consumer product bases or when exposed to ambient air. Entrapment of perfume ingredients in a water-soluble matrix makes it possible to protect them from those influences, which allows conserving the exact composition of a perfume developed by a formulator and/or perfumer. The perfume ingredients can be released from the matrix by a dual mode of activation: Either under moisture (e.g. atmospheric moisture or perspiration) and/or mechanical activation (e.g. friction).

The alcohol can be selected from the group consisting of ethanol, isopropanol, ethylene glycol, propylene glycol and glycerol, preferably ethanol and isopropanol, even more preferably ethanol.

The water-soluble matrix can comprise at least one material selected from the group consisting of starch, in particular water-soluble modified starch, maltodextrin, mannitol, chitosan, gum Arabic, alginate, cellulose, pectins, gelatin, polyvinyl alcohol and mixtures thereof. The resulting perfume formulations are facile and cost-effective in manufacture, for instance by spray-drying. They are compatible with standard propellants in compositions formulated as an aerosol and resistant to compression. Furthermore, they are prepared of naturally-based materials, which are non-toxic and biodegradable. Such formats therefore have an increased consumer-appeal.

When the starch is a water-soluble modified starch, such starch can be made from raw starch or pre-gelatinized starch. It can be derived from tubers, legumes, cereals and grains, for example corn starch, wheat starch, rice starch, waxy corn starch, oat starch, cassava starch, waxy barley starch, waxy rice starch, sweet rice starch, amioca starch, potato starch, tapioca starch and mixtures thereof.

The water-soluble modified starch can be selected from the group consisting of bleached starch, hydroxypropyl starch, hydroxypropyl distarch phosphate, dydroxypropyl distarch glycerol, acetylated distarch phosphate, starch acetate esterified with acetic anhydride, starch acetate esterified with vinyl acetate, acetylated distarch adipate, acetylated distarch glycerol, starch sodium octenyl succinate and mixtures thereof.

Water-soluble modified starches have emulsifying and emulsion-stabilizing capacity. They have the ability to entrap perfume droplets in the form of oilin-water emulsions due to the hydrophobic character of the starch modifying agent. The modified starches as described herein above bring numerous advantages including high emulsification and entrapping performance, low viscosity, even at high solids content, and excellent oxidation resistance to ensure good perfume preservation and stabilization of sensitive ingredients.

When the water-soluble matrix comprises a water-soluble modified starch, it can additionally comprise a material selected from the group consisting of maltodextrin, mannitol and mixtures thereof. Maltodextrin and mannitol both increase the glass transition temperature of the matrix. Furthermore, maltodextrin is a film forming agent.

Maltodextrins are characterized by their dextrin equivalent (DE). The higher the DE, the lower is the molecular weight of the maltodextrin. In the context of the present invention, maltodextrin having different DE may be combined to provide optimized encapsulation properties. Without being bound by any theory, it is supposed that mixtures of low and high DE maltodextrins improve the packing of the water-soluble matrix.

Further to the materials stated herein above, the water-soluble matrix can additionally comprise a hemicellulose. In the context of the present invention, the expression “hemicellulose” is to be understood as a polysaccharide selected from the group consisting of glucans, in particular xyloglucans, mannans, in particular glucomannans, and xylans, in particular arabinoxylans and glucuronoxylans.

It has been found that addition of a hemicellulose to a water-soluble matrix, in particular a starch matrix, leads to a modification of the matrix, improving its perfume release properties under moisture and mechanical (e.g. friction) activation.

The hemicellulose is preferably a xyloglucan, in particular a xyloglucan obtainable from tamarind seeds. Xyloglucans are the most abundant hemicellulose in the primary walls of non-graminaceous plants, often comprising 20 wt.-% of the dry mass of the wall. A xyloglucan has a backbone composed of 1,4-linked β-D-glucose residues. Up to 75% of the backbone residues are substituted at C6 with mono-, di-, or trisaccharide sidechains. Preferably, the hemicellulose is a xyloglucan obtainable from tamarind seeds, in particular obtained from tamarind seeds, also known as “tamarind kernel powder” or “tamarind gum”. In tamarind gum, the side chains consist of one or two α-D-xylopyranosyl units, optionally capped with β-D-galactopyranosyl, α-L-arabinofuranosyl or β-D-xylopyranosyl.

In preferred embodiments of the present invention, the water-soluble matrix is in particulate form.

When the water-soluble matrix is in particulate form, it can have a poured powder density of 300-600 g/l, preferably 325-500 g/l, more preferably 350-450 g/l. By virtue of their low poured powder density (loose bulk density), such particles do not sediment or sediment only very little. They are therefore compatible with propellant gases and are resistant to compression so as to be able to be formulated in an aerosol device without being damaged.

Determination of the poured powder density is performed at room temperature (25° C.) and under normal atmospheric conditions (1013.25 hPa) using a 100 ml measuring cylinder. The measuring cylinder is weighed empty and then filled with a volume of 100 ml of poured powder, without tapping. The difference in mass between the empty measuring cylinder and the cylinder filled with 100 ml of powder gives the poured powder density.

The particles can have a number-mean diameter of 1-30 μm, preferably 2-20 μm, more preferably 3-10 μm, and a volume-mean diameter of 5-150 μm, preferably 10-100 μm, more preferably 20-50 μm. The particle size distribution can be determined by dry dispersion laser diffraction.

In the present context, the expression “anhydrous composition” is to be understood as a composition that has a water content of less than 5 wt.-%, relative to the total weight of the composition. In preferred embodiments the water content is less than 2 wt.-%, more preferably less than 1 wt.-%, even more preferably less than 0.5 wt.-%, even still more preferably less than 0.1 wt.-%, relative to the total weight of the composition.

The at least one cosmetically acceptable excipient can be selected from the group consisting of preservatives, antioxidants, chelating agents, sunscreen agents, vitamins, dyes, hair coloring agents, proteins, amino acids, plant extracts, humectants, oils, emollients, lubricants, butters, penetrants, thickeners, viscosity modifiers, polymers, resins, hair fixatives, film formers, surfactants, detergents, emulsifiers, opacifying agents, volatiles, liquid vehicles, carriers, salts, pH adjusting agents, neutralizing agents, buffers, hair conditioning agents, anti-static agents, anti-frizz agents, anti-dandruff agents, hair waving agents, hair straightening agents, relaxers, absorbents, deodorant agents, antiperspirant agents, anti-caking agents and combinations thereof.

In a preferred embodiment of the present invention, in particular in a dry shampoo, the at least one cosmetically acceptable excipient is an absorbent, preferably in combination with further cosmetically acceptable excipients, such as anti-caking agents and/or preservatives and/or carriers. The absorbent has the function to absorb dirt, oil and/or sebum form the hair.

The absorbent is preferably selected from starches, in particular form corn starch, wheat starch, rice starch, oat starch, cassava starch, amioca starch, tapioca starch and potato starch; clays, in particular a smectite clay (such as hectorite) and Kaolin; Oryza sativa (Rice) hull powder; and mixtures thereof. The absorbent can also be selected from starch derivatives, such as dimethylimidazolidinone rice starch, corn starch octenylsuccinate and aluminum starch octenylsuccinate.

The anti-caking agent can be selected from the group consisting of calcium carbonate, calcium phosphate, magnesium phosphate, silicon dioxide, magnesium oxide, calcium oxide, magnesium hydroxide, calcium hydroxide, calcium silicate, calcium citrate, monocalcium orthophosphate monohydrate, aluminum dimyristate, aluminum isostearates/myristates, aluminum myristate, aluminum myristates/palmitates, calcium myristate, magnesium myristate and zinc myristate.

The preservative can be selected from the group consisting of potassium sorbate, sodium sulphite, sodium benzoate and benzoic acid.

In a preferred embodiment of the present invention, in the carrier is isopropyl myristate.

In another preferred embodiment of the present invention, in particular in an antiperspirant and/or a deodorant, the at least one cosmetically acceptable excipient is an antiperspirant agent and/or a deodorant agent.

The term “antiperspirant agent” means a compound which, by itself, has the effect of reducing the flow of sweat and/or of reducing the sensation of moisture on the skin associated with human sweat and/or of partially or totally absorbing human sweat. Among the antiperspirant active agents that may be mentioned are aluminum and/or zirconium salts, such as aluminum chlorohydrate, aluminum chlorohydrex, aluminum chlorohydrex PEG, aluminum chlorohydrex PG, aluminum dichlorohydrate, aluminum dichlorohydrex PEG, aluminum dichlorohydrex PG, aluminum sesquichlorohydrate, aluminum sesquichlorohydrex PEG, aluminum sesquichlorohydrex PG, aluminum sulfate, aluminum zirconium octachlorohydrate, aluminum zirconium pentachlorohydrate, aluminum zirconium tetrachlorohydrate and aluminum zirconium trichlorohydrate.

The term “deodorant agent” refers to any substance that is capable of masking, absorbing, improving and/or reducing the unpleasant odor resulting from the decomposition of human sweat by bacteria.

The deodorant agents may be bacteriostatic agents or bactericides that act on odor microorganisms, such as 2,4,4′-trichloro-2′-hydroxydiphenyl ether, 2,4-dichloro-2′-hydroxydiphenyl ether, 3′,4′,5′-trichlorosalicylanilide, 1-(3′,4′-dichlorophenyl)-3-(4′-chlorophenyl)urea or 3,7,11-trimethyldodeca-2,5,10-trienol; quaternary ammonium salts such as cetyltrimethylammonium salts, cetylpyridinium salts; polyols such as those of glycerol type, 1,3-propanediol, 1,2-decanediol, glycerol derivatives, for instance caprylic/capric glycerides, glyceryl caprylate or caprate, polyglyceryl-2 caprate, biguanide derivatives, for instance polyhexamethylene biguanide salts; chlorhexidine and salts thereof; 4-phenyl-4,4-dimethyl-2-butanol; cyclodextrins; chelating agents such as tetrasodium glutamate diacetate, EDTA (ethylenediaminetetraacetic acid) and DPTA (1,3-diaminopropanetetraacetic acid).

Mention may also be made of zinc salts, such as zinc salicylate, zinc phenolsulfonate, zinc pyrrolidonecarboxylate (more commonly known as zinc pidolate), zinc sulfate, zinc chloride, zinc lactate, zinc gluconate, zinc ricinoleate, zinc glycinate, zinc carbonate, zinc citrate, zinc chloride, zinc laurate, zinc oleate, zinc orthophosphate, zinc stearate, zinc tartrate, zinc acetate or mixtures thereof; odor absorbers such as zeolites, especially silver-free metal zeolites, cyclodextrins, metal oxide silicates; metal oxide particles modified with a transition metal; aluminosilicates; chitosan-based particles; sodium bicarbonate; salicylic acid and derivatives thereof such as 5-n-octanoylsalicylic acid; alum; and triethyl citrate.

The emollient can be selected from the group consisting of butyl stearate, isocetyl stearate, isopropyl myristate, isopropyl palmitate and isopropyl stearate.

The composition can further comprise a propellant. The propellant can be selected from the group consisting of dimethyl ether, volatile hydrocarbons such as propane, n-butane, isobutane, n-pentane and isopentane, chlorinated and/or fluorinated hydrocarbons, and mixtures thereof.

Preferred propellants are the following:
A-40: Mixture of Hydrocarbons;
A-46: Mixture of Hydrocarbons;
HFC-152A: 1,1-Difluoroethane;
HFC-134A: 1,1,1,2-Tetrafluoroethane;
DME: Dimethyl Ether;

When the propellant is chosen from volatile hydrocarbons, it is preferably chosen from propane, n-butane, isobutane, n-pentane, isopentane, and mixtures thereof.

Carbon dioxide, nitrous oxide, nitrogen or compressed air may also be used as propellant gas.

The amount of propellant used in a composition according to the present invention can be 40-95 wt.-%, preferably 50-90 wt.-%, more preferably 60-85 wt.-%, relative to the total weight of the composition.

The at least one perfume ingredient can belong to different classes of organic compounds, as varied as alcohols, ketones, esters, ethers, acetates, terpene hydrocarbons, nitrogenous or sulphurous heterocyclic compounds and essential oils, which can be of natural or synthetic origin. Many of these perfume ingredients are listed in reference texts, such as S. Arctander, Perfume and Flavor Chemicals, 1994, Montclair, New Jersey, USA.

Preferably, the at least one perfume ingredient has a boiling point determined at the normal standard pressure of 1013.25 hPa of 275° C. or lower and an odor detection threshold of less than or equal to 50 parts per billion (ppb).

In a particular embodiment of the present invention, the least one perfume ingredient is selected from the group consisting of ACETOPHENONE EXTRA (1-phenylethanone); ADOXAL (2,6,10-trimethylundec-9-enal); AGRUMEX (2-(tert-butyl)cyclohexyl acetate); ALCOHOL C 6 HEXYLIC (hexan-1-ol); ALDEHYDE C 10 DECYLIC (decanal); ALDEHYDE C 11 MOA (2-methyldecanal); ALDEHYDE C 11 UNDECYLENIC (undec-10-enal); ALDEHYDE C 110 UNDECYLIC (undecanal); ALDEHYDE C 12 LAURIC (dodecanal); ALDEHYDE C 12 MNA PURE (2-methylundecanal); ALDEHYDE C 6 HEXYLIC FOOD GRADE (hexan-1-al); ALDEHYDE C 8 OCTYLIC FOOD GRADE (octanal); ALDEHYDE C 9 ISONONYLIC (3,5,5-trimethylhexanal); ALDEHYDE C 9 NONYLIC FOOD GRADE (nonanal); ALDEHYDE ISO C 11 ((E)-undec-9-enal); ALLYL AMYL GLYCOLATE (prop-2-enyl 2-(3-methylbutoxy)acetate); ALLYL CAPROATE (prop-2-enyl hexanoate); ALLYL CYCLOHEXYL PROPIONATE (prop-2-enyl 3-cyclohexylpropanoate); ALLYL OENANTHATE (prop-2-enyl heptanoate); AMBERKETAL (3,8,8,11a-tetramethyldodecahydro-1H-3,5aepoxynaphtho[2,1-c]oxepine); AMBRETTOLIDE ((Z)-oxacycloheptadec-10-en-2-one); AMBROFIX ((3aR,5aS,9aS,9bR)-3a,6,6,9a-tetramethyl-2,4,5,5a,7,8,9,9b-octahydro-1H-benzo[e][1]benzofuran); AMYL BUTYRATE (pentyl butanoate); AMYL CINNAMIC ALDEHYDE ((Z)-2-benzylideneheptanal); AMYL SALICYLATE (pentyl 2-hydroxybenzoate); ANETHOLE ((E)-1-methoxy-4-(prop-1-en-1-yl)benzene); ANISYL ACETATE (4-methoxybenzyl acetate); APHERMATE (1-(3,3-dimethylcyclohexyl)ethyl formate); AUBEPINE PARA CRESOL (4-methoxybenzaldehyde); AURANTIOL ((E)-methyl 2-((7-hydroxy-3,7-dimethyloctylidene)amino)benzoate); BENZALDEHYDE (benzaldehyde); BENZYL ACETATE (benzyl acetate); BENZYL ACETONE (4-phenylbutan-2-one); BENZYL ALCOHOL (phenylmethanol); BENZYL BENZOATE (benzyl benzoate); BENZYL CINNAMATE (benzyl 3-phenylprop-2-enoate); BENZYL SALICYLATE (benzyl 2-hydroxybenzoate); BICYCLO NONALACTONE (octahydro-2H-chromen-2-one); BORNEOL CRYSTALS ((1S,2S,4S)-1,7,7-trimethylbicyclo[2.2.1]heptan-2-ol); BORNYL ACETATE ((2S,4S)-1,7,7-trimethylbicyclo[2.2.1]heptan-2-yl acetate); BOURGEONAL (3-(4-(tert-butyl)phenyl)propanal); BUTYL ACETATE (butyl acetate); BUTYL CYCLOHEXYL ACETATE PARA (4-(tert-butyl)cyclohexyl acetate); CAMPHOR ((1S,4S)-1,7,7-trimethylbicyclo[2.2.1]heptan-2-one); CARVONE LAEVO ((5R)-2-methyl-5-prop-1-en-2-ylcyclohex-2-en-1-one); CEDRYL METHYL ETHER ((1R,6S,8aS)-6-methoxy-1,4,4,6-tetra-methyloctahydro-1H-5,8a-methanoazulene); CETONE V ((E)-1-(2,6,6-tri-methylcyclohex-2-en-1-yl)hepta-1,6-dien-3-one); CINNAMIC ALCOHOL SYNTHETIC ((E)-3-phenylprop-2-en-1-ol); CINNAMIC ALDEHYDE ((2E)-3-phenylprop-2-enal); CINNAMYL ACETATE ((E)-3-phenylprop-2-en-1-yl acetate); CIS-3-HEXENOL ((Z)-hex-3-en-1-ol); CIS JASMONE ((Z)-3-methyl-2-(pent-2-en-1-yl)cyclopent-2-enone); CITRAL ((E)-3,7-dimethylocta-2,6-dienal); CITRONELLAL (3,7-dimethyloct-6-enal); CITRONELLOL (3,7-dimethyloct-6-en-1-ol); CITRONELLYL ACETATE (3,7-dimethyloct-6-en-1-yl acetate); CITRONELLYL FORMATE (3,7-dimethyloct-6-en-1-yl formate); CITRONELLYL NITRILE (3,7-dimethyloct-6-enenitrile); CLONAL (dodecaneni-trile); CORANOL (4-cyclohexyl-2-methylbutan-2-ol); CORYLONE DRIED (2-hydroxy-3-methylcyclopent-2-enone); COSMONE ((Z)-3-methylcyclo-tetradec-5-enone); COUMARIN PURE CRYSTALS (2H-chromen-2-one); CRESYL METHYL ETHER PARA (1-methoxy-4-methylbenzene); CUMIN NITRILE (4-isopropylbenzonitrile); CYCLAMEN ALDEHYDE EXTRA (3-(4-iso-propylphenyl)-2-methylpropanal); DAMASCENONE ((E)-1-(2,6,6-trimethyl-cyclohexa-1,3-dien-1-yl)but-2-en-1-one); DAMASCONE ALPHA ((E)-1-(2,6,6-trimethylcyclohex-2-en-1-yl)but-2-en-1-one); DECALACTONE GAMMA (5-hexyloxolan-2-one); DECENAL-4-TRANS ((E)-dec-4-enal); DIHYDRO ANETHOLE (1-methoxy-4-propylbenzene); DIHYDRO MYRCENOL (2,6-dimethyloct-7-en-2-ol); DIMETHYL BENZYL CARBINYL ACETATE (2-me-thyl-1-phenylpropan-2-yl acetate); DIMETHYL BENZYL CARBINYL BUTYRATE (2-methyl-1-phenylpropan-2-yl butanoate); DIMETOL (2,6-dimethylheptan-2-ol); DIPHENYL OXIDE (oxydibenzene); DODECENAL ((E)-dodec-2-enal); EBANOL ((E)-3-methyl-5-(2,2,3-trimethylcyclopent-3-en-1-yl)pent-4-en-2-ol); ESTERLY (ethyl cyclohexyl carboxylate); ETHYL ACETATE (ethyl acetate); ETHYL ACETOACETATE (ethyl 3-oxobutanoate); ETHYL CINNAMATE (ethyl 3-phenylprop-2-enoate); ETHYL HEXANOATE (ethyl hexanoate); ETHYL LINALOOL ((E)-3,7-dimethylnona-1,6-dien-3-ol); ETHYL MALTOL (2-ethyl-3-hydroxy-4H-pyran-4-one); ETHYL METHYL-2-BUTYRATE (ethyl 2-methylbutanoate); ETHYL OENANTHATE (ethyl heptanoate); ETHYL VANILLIN (3-ethoxy-4-hydroxybenzaldehyde); ETHYLENE BRASSYLATE (1,4-dioxacycloheptadecane-5,17-dione); EUCALYPTOL NATURAL ((1s,4s)-1,3,3-trimethyl-2-oxabicyclo[2.2.2]octane); EUGENOL (4-allyl-2-methoxy-phenol); EVERNYL (methyl 2,4-dihydroxy-3,6-dimethylbenzoate); FENCHYL ALCOHOL ((1S,2R,4R)-1,3,3-trimethylbicyclo[2.2.1]heptan-2-ol); FENNALDEHYDE (3-(4-methoxyphenyl)-2-methylpropanal); FLORHYDRAL (3-(3-isopropylphenyl)butanal); FLOROSA HC (tetrahydro-4-methyl-2-(2-methylpropyl)-2H-pyran-4-ol); FRESKOMENTHE (2-(sec-butyl)cyclohexa-none); FRUTONILE (2-methyldecanenitrile); GALBANONE PURE (1-(5,5-di-methylcyclohex-1-en-1-yl)pent-4-en-1-one); GERANIOL ((E)-3,7-dime-thylocta-2,6-dien-1-ol); GERANYL ACETATE ((E)-3,7-dimethylocta-2,6-dien-1-yl acetate); GERANYL ACETONE ((E)-6,10-dimethylundeca-5,9-dien-2-one); HABANOLIDE ((E)-oxacyclohexadec-12-en-2-one); HEDIONE (methyl 3-oxo-2-pentylcyclopentaneacetate); HELIOTROPINE CRYSTALS (benzo[d][1,3]dioxole-5-carbaldehyde); HEXENAL-2-TRANS ((E)-hex-2-enal); HEXENOL-3-CIS ((Z)-hex-3-en-1-ol); HEXENYL-3-CIS ACETATE ((Z)-hex-3-en-1-yl acetate); HEXENYL-3-CIS ISOBUTYRATE ((Z)-hex-3-en-1-yl 2-methylpropanoate); HEXENYL-3-CIS SALICYLATE ((Z)-hex-3-en-1-yl 2-hydroxybenzoate); HEXYL ACETATE (hexyl acetate); HEXYL CINNAMIC ALDEHYDE ((E)-2-benzylideneoctanal); HEXYL ISOBUTYRATE (hexyl 2-methylpropanoate); HEXYL SALICYLATE (hexyl 2-hydroxybenzoate); HYDROXYCITRONELLAL (7-hydroxy-3,7-dimethyloctanal); INDOLE PURE (1H-indole); IONONE BETA ((E)-4-(2,6,6-trimethylcyclohex-1-en-1-yl)but-3-en-2-one); IRISONE ALPHA ((E)-4-(2,6,6-trimethylcyclohex-2-en-1-yl)but-3-en-2-one); ISOAMYL ACETATE EXTRA (3-methylbutyl acetate); ISOEUGENOL ((E)-2-methoxy-4-(prop-1-en-1-yl)phenol); ISOMENTHONE DL (2-isopropyl-5-methylcyclohexanone); ISOPROPYL METHYL-2-BUTYRATE (isopropyl 2-methylbutanoate); ISORALDEINE ((E)-3-methyl-4-(2,6,6-tri-methylcyclohex-2-en-1-yl)but-3-en-2-one); JASMONE CIS ((Z)-3-methyl-2-(pent-2-en-1-yl)cyclopent-2-enone); JASMONYL (3-butyl-5-methyltetrahy-dro-2H-pyran-4-yl acetate); JASMOPYRANE FORTE (3-pentyltetrahydro-2H-pyran-4-yl acetate); LIFFAROME ((Z)-hex-3-en-1-yl methyl carbonate); LILIAL (3-(4-(tert-butyl)phenyl)-2-methylpropanal); LIMONENE, LIMONENE, or LAEVO, LIMONENE DEXTRO (1-methyl-4-prop-1-en-2-yl-cyclohexene); LINALOOL OXIDE (2-(5-methyl-5-vinyltetrahydrofuran-2-yl)propan-2-ol); LINALOOL (3,7-dimethylocta-1,6-dien-3-ol); LINALYL ACETATE (3,7-dime-thylocta-1,6-dien-3-yl acetate); MAHONIAL ((4E)-9-hydroxy-5,9-dimethyl-4-decenal); MALTOL (3-hydroxy-2-methyl-4H-pyran-4-one); MANZANATE (ethyl 2-methylpentanoate); MAYOL ((4-isopropylcyclohexyl)methanol); MEFROSOL (3-methyl-5-phenylpentan-1-ol); MELONAL (2,6-dimethylhept-5-enal); MENTHOL, MENTHOL LAEVO, or MENTHOL RACEMIC (2-isopropyl-5-methylcyclohexanol); MENTHONE, ISOMENTHONE, MENTHONE LAEVO, or MENTHONE RACEMIC (2-isopropyl-5-methylcyclohexanone); METHYL ANTHRANILATE EXTRA (methyl 2-aminobenzoate); METHYL BENZOATE (methyl benzoate); METHYL CINNAMATE (methyl 3-phenylprop-2-enoate); METHYL DIANTILIS (2-ethoxy-4-(methoxymethyl)phenol); METHYL DIHYDRO ISOJASMONATE (methyl 2-hexyl-3-oxocyclopentane-1-carboxylate); METHYL HEPTENONE (6-methylhept-5-en-2-one); METHYL LAITONE (8-methyl-1-oxaspiro[4.5]decan-2-one); METHYL OCTYNE CARBONATE (methyl non-2-ynoate); METHYL SALICYLATE (methyl 2-hydroxybenzoate); MUSCENONE ((Z)-3-methylcyclopentadec-5-enone); MYRALDENE (4-(4-methylpent-3-en-1-yl)cyclohex-3-enecarbaldehyde); MYRCENE (7-methyl-3-methyleneocta-1,6-diene); MYSTIKAL (2-methylundecanoic acid); NECTARYL (2-(2-(4-methylcyclohex-3-en-1-yl)propyl)cyclopentanone); NEOFOLIONE ((E)-methyl non-2-enoate); NEROLEX ((2Z)-3,7-dimethylocta-2,6-dien-1-ol); NEROLIDOL ((E)-3,7,11-trimethyldodeca-1,6,10-trien-3-ol); NEROLINE CRYSTALS (2-ethoxynaphthalene); NERYL ACETATE HC ((Z)-3,7-dimethylocta-2,6-dien-1-yl acetate); NIRVANOLIDE ((E)-13-methyloxacy-clopentadec-10-en-2-one); NONADIENAL ((2E,6Z)-nona-2,6-dienal); NONADIENOL-2,6 ((2Z,6E)-2,6-nonadien-1-ol); NONALACTONE GAMMA (5-pentyloxolan-2-one); NONENAL-6-CIS ((Z)-non-6-enal); NONENOL-6-CIS ((Z)-non-6-en-1-ol); NOPYL ACETATE (2-(6,6-dimethylbicyclol[3.1.1]hept-2-en-2-yl)ethyl acetate); NYMPHEAL (3-(4-(2-methylpropyl)-2-methylphenyl)propanal); OCTANONE-2 (octan-2-one); ORANGER CRYSTALS (1-(2-naphtalenyl)-ethanone); PANDANOL ((2-methoxyethyl)benzene); PEACH PURE (5-heptyldihydrofuran-2(3H)-one); PELARGOL (3,7-dime-thyloctan-1-ol); PHARAONE (2-cyclohexylhepta-1,6-dien-3-one); PHENOXY ETHYL ISOBUTYRATE (2-(phenoxy)ethyl 2-methylpropanoate); PHENYL ACETALDEHYDE (2-phenyl-ethanal); PHENYL ETHYL ACETATE (2-phenylethyl acetate); PHENYL ETHYL ALCOHOL (2-phenylethanol); PHENYL ETHYL ISOBUTYRATE (2-phenylethyl 2-methylpropanoate); PHENYL ETHYL PHENYL ACETATE (2-phenylethyl 2-phenylacetate); PHENYL PROPYL ALCOHOL (3-phenylpropan-1-ol); PINENE BETA (6,6-dimethyl-2-methylenebicy-clo[3.1.1]heptane); PINOACETALDEHYDE (3-(6,6-dimethylbicy-clo[3.1.1]hept-2-en-2-yl)propanal); POMAROSE ((2E,5E)-5,6,7-trime-thylocta-2,5-dien-4-one); PRECYCLEMONE B (1-methyl-4-(4-methylpent-3-en-1-yl)cyclohex-3-enecarbaldehyde); PRENYL ACETATE (3-methylbut-2-en-1-yl acetate); RASPBERRY KETONE (4-(4-hydroxyphenyl)butan-2-one); ROSE OXIDE (4-methyl-2-(2-methylprop-1-en-1-yl)tetrahydro-2H-pyran); ROSYRANE SUPER (4-methyl-2-phenyl-3,6-dihydro-2H-pyran); SANDALORE EXTRA (3-methyl-5-(2,2,3-trimethylcyclopent-3-en-1-yl)pentan-2-ol); SCENTAURUS CLEAN (ethyl (Z)-2-acetyl-4-methyltridec-2-enoate); SCENTAURUS JUICY (4-(dodecylthio)-4-methylpentan-2-one); SILVANONE SUPRA (cyclopentadecanone, hexadecanolide); STYRALLYL ACETATE (1-phenylethyl acetate); SUPER MUGUET ((E)-6-ethyl-3-methyloct-6-en-1-ol); SYLKOLIDE ((E)-2-((3,5-dimethylhex-3-en-2-yl)oxy)-2-methylpropyl cyclo-propanecarboxylate); TERPINENE GAMMA (1-methyl-4-propan-2-ylcyclo-hexa-1,4-diene); TERPINEOL ALPHA (2-(4-methyl-1-cyclohex-3-enyl)pro-pan-2-ol); TERPINEOL PURE (2-(4-methylcyclohex-3-en-1-yl)propan-2-ol); TERPINOLENE (1-methyl-4-(propan-2-ylidene)cyclohex-1-ene); TERPINYL ACETATE (2-(4-methyl-1-cyclohex-3-enyl)propan-2-yl acetate); TETRAHYDRO LINALOOL (3,7-dimethyloctan-3-ol); THIBETOLIDE (oxacyclo-hexadecan-2-one); THYMOL (2-isopropyl-5-methylphenol); TOSCANOL (1-(cyclopropylmethyl)-4-methoxybenzene); TRIDECENE-2-NITRILE ((E)-tridec-2-enenitrile); TRIFERNAL (3-phenylbutanal); TROPIONAL (3-(benzo[d][1,3]dioxol-5-yl)-2-methylpropanal); METHYL NONYL KETONE (undecan-2-one); UNDECATRIENE ((3E,5Z)-undeca-1,3,5-triene); UNDECAVERTOL ((E)-4-methyldec-3-en-5-ol); VANILLIN (4-hydroxy-3-methoxybenzaldehyde); VELVIONE ((Z)-cyclohexadec-5-enone); VIOLET NITRILE ((2E,6Z)-nona-2,6-dienenitrile); and YARA YARA (2-methoxynaph-talene).

The above-mentioned ingredients have all been identified as being suitable for entrapment in a water-soluble matrix, especially with respect to their physical and chemical properties, such as lipophilicity, molecular size and reactivity towards matrix materials. Furthermore, these ingredients fulfill high ecological standards, in particular with respect to biodegradation.

In a preferred embodiment, the amount of the encapsulated perfume for-mulation, i.e. the amount of the least one perfume ingredient entrapped in the water-soluble matrix and the matrix material, is 0.01-5.0 wt.-%, preferably 0.05-2.0 wt.-%, more preferably 0.1-1.0 wt.-%, relative to the total weight of the composition.

In addition to at least one perfume ingredient that is entrapped in a water-soluble matrix, compositions according to the present invention can also comprise free perfume oil. The amount of free perfume oil in a composition according to the present invention can be 0.01-2.0 wt.-%, preferably 0.02-1.0 wt.-%, more preferably 0.05-0.5 wt.-%, relative to the total weight of the composition.

In addition to the above-mentioned perfume ingredients, compositions according to the present invention can also comprise a malodor counteractant. The malodor counteractant can be selected from the following classes:
Perfume compositions designed to counteract malodors;
Reactive perfume ingredients (such as α-β-unsaturated carbonyl compounds, e.g. dialkylfumarates or damascones);
Other chemical systems (such as alkylene carbonates, zinc riccinoleate, zinc polyitaconate, zinc oxide, cysteine or imines);
Adsorbents (such as zeolites, active carbon, aluminum oxides or silica gels);
Host-guest compounds (such as cyclodextrins).

In a further preferred embodiment of the present invention, the amount the at least one cosmetically acceptable excipient in the composition is 0.1-99.9 wt.-%, preferably 0.5-80%, more preferably 1-40 wt.-%, even more preferably 2-30 wt.-%, even still more preferably 5-25 wt.-%, relative to the total weight of the composition.

A second aspect of the present invention refers to a consumer product comprising a composition as described herein above. In the present context, the term “consumer product” means any manufactured product intended to be used or consumed in the form in which it is sold and which is not intended for a subsequent manufacture or modification.

Without any limitation, consumer products according to the present invention may be cosmetic products, also including cosmetic formulations for car-ing for and/or for the hygiene of and/or for making up the skin, the lips, the nails, the eyelashes, the eyebrows, the hair or the scalp; dermatological products; fragrance products; pharmaceutical products; products for veteri-nary use, especially animal hygiene and/or care products.

In preferred embodiments of the present invention, the consumer product is selected from the group consisting of dry shampoos, hair styling products, deodorants and antiperspirants.

The consumer product can be in the form of a spray, in particular an aerosol spray. The consumer product can be selected from the group consisting of dry shampoo sprays (in particular dry shampoo sprays for humans or for pets, e.g. dogs or cats), hair sprays, styling sprays, deodorant sprays, antiperspirant sprays and cat litter refresher sprays.

When the consumer product is in the form of an aerosol spray, the amount the at least one cosmetically acceptable excipient in the composition can be 1-40 wt.-%, preferably 2-30 wt.-%, more preferably 5-25 wt.-%, relative to the total weight of the composition.

A third aspect of the present invention relates to a method for treating a keratinous surface, in particular hair, preferably scalp hair. The method comprises the step of applying to the keratinous surface a composition as described herein above, in particular in a consumer product as described herein above. Preferably, the method does not comprise a rinsing step.

Further advantages and particular features of the present invention become apparent from the following description of several examples.

### EXAMPLE 1: ENTRAPMENT OF PERFUME IN WATER-SOLUBLE MATRIX

Perfume oil can be entrapped in a water-soluble matrix as follows:

Tap water (50.0 g) is weighted into a stainless steel beaker. Starch sodium octenyl succinate E1450 (20.0 g), starch modified Hi-Cap 100 (3.0 g) and maltodextrin Glucidex IT-19 (6.0 g) are subsequently weighted into the same beaker. The resulting mixture is first manually stirred with a stainless steel rod and then homogenized with an IKA T25 Ultra-Turrax Homogenizer at 13,500 rpm to obtain a homogeneous solution. To this resulting mixture, perfume oil (20.0 g) is added. High shear mixing is then carried out for 20-30 min at 22,000-24,000 rpm using the same Homogenizer to produce an emulsion. The droplet size is controlled by dynamic light scattering to be between 0.5 and 2 μm.

The emulsion is subjected to spray drying using a LabPlant SD-06 Spray Dryer. The spray drying process parameters are as follows:
Inlet Temperature: 190° C.
Outlet Temperature: 90° C.
Peristaltic pump speed: 485 mL/h
Air flow rate: 3.7 m/s

The resulting spray dried powder typically has a poured powder density of 400 g/l. The number-mean diameter of the particles (weighted arithmetic mean {numeric}) is 5 μm and the volume mean-diameter (D[4;3]{volumic}) is 40 μm.

The powder is mixed with silicon dioxide Aerosil 200 (1.0 g) in a closed mixing vessel.

### EXAMPLE 2: ENTRAPMENT OF PERFUME IN WATER-SOLUBLE MATRIX

Alternatively, perfume oil can be entrapped in a water-soluble matrix as follows:

Tap water (55.0 g) is weighted into a stainless steel beaker. Starch sodium octenyl succinate E1450 (18.7 g), starch modified Hi-Cap 100 (2.2 g), maltodextrin Glucidex IT-19 (5.3 g) and tamarind kernel powder (0.5 g) are subsequently weighted into the same beaker. The resulting mixture is first manually stirred with a stainless steel rod and then homogenized with an IKA T25 Ultra-Turrax Homogenizer at 13,500 rpm to obtain a homogeneous solution. To this resulting mixture, perfume oil (17.8 g) is added. High shear mixing is then carried out for 20-30 min at 22,000-24,000 rpm using the same Homogenizer to produce an emulsion. The droplet size is controlled by dynamic light scattering to be between 0.5 and 2 μm.

The emulsion is subjected to spray drying using a LabPlant SD-06 Spray Dryer. The spray drying process parameters are as follows:
Inlet Temperature: 190° C.
Outlet Temperature: 90° C.
Peristaltic pump speed: 485 mL/h
Air flow rate: 3.7 m/s

The resulting spray dried powder typically has a poured powder density of 400 g/l. The number-mean diameter of the particles (weighted arithmetic mean {numeric}) is 5 μm and the volume mean-diameter (D[4;3]{volumic}) is 40 μm.

The powder is mixed with silicon dioxide Aerosil 200 (0.5 g) in a closed mixing vessel.

### EXAMPLE 3: ENTRAPMENT OF PERFUME IN WATER-SOLUBLE MATRIX

As a further alternative, perfume oil can be entrapped in a water-soluble matrix as follows:

Tap water (45.0 g) is weighted into a stainless steel beaker. Starch sodium octenyl succinate E1450 (21.9 g), mannitol 60 (5.5 g) and tamarind kernel powder (0.5 g) are subsequently weighted into the same beaker. The resulting mixture is first manually stirred with a stainless steel rod and then homogenized with an IKA T25 Ultra-Turrax Homogenizer at 13,500 rpm to obtain a homogeneous solution. To the resulting mixture, perfume oil (27.3 g) is added. High shear mixing is then carried out for 20-30 min at 22,000-24,000 rpm using the same Homogenizer to produce an emulsion. The droplet size is controlled by dynamic light scattering to be between 0.5 and 2 μm.

The emulsion is subjected to spray drying using a LabPlant SD-06 Spray Dryer. The spray drying process parameters are as follows:
Inlet Temperature: 190° C.
Outlet Temperature: 90° C.
Peristaltic pump speed: 485 mL/h
Air flow rate: 3.7 m/s

The resulting spray dried powder typically has a poured powder density of 400 g/l. The number-mean diameter of the particles (weighted arithmetic mean {numeric}) is 5 μm and the volume mean-diameter (D[4;3]{volumic}) is 40 μm.

The powder is mixed with silicon dioxide Aerosil 200 (0.5 g) in a closed mixing vessel.

### EXAMPLE 4: PREPARATION OF DRY SHAMPOOS ACCORDING TO THE PRESENT INVENTION

Aerosol sprays containing a dry shampoo according to the present invention were prepared as follows:
A blank commercially available aerosol can, which is suitable for dry shampoo applications, was placed on a tared weight balance. All components of the dry shampoo composition, except the propellant, were added into the aerosol can, as specified in below Tables 1 to 3. After each addition, the can was slightly swirled in a circular motion to mix in order to ensure homoge-neity. Once all components were added, a mounting cup/valve was placed on top of the can and crimped firmly according to the valve specifications using the appropriate equipment. The can was then filled with the propellant in a secure and safe area designed to aerosolize cans. The filled can was placed in a water bath to ensure proper crimping and no leakage. Once this quality check was completed, an actuator valve was placed on the mounting cup. The aerosol cans were stored at room temperature for 2 weeks before testing.

TABLE 1
Dry Shampoo Compositions with Free Perfume Oil

| | 4a | 4b | 4c |
|---|---|---|---|
| Example | [wt.-%] | [wt.-%] | [wt.-%] |
| Ethanol ^{a} | 0 | 5 | 20 |
| Dry Flo TS ^{b} | 5 | 5 | 5 |
| Isopropyl Myristate | 14.8 | 9.8 | 4.8 |
| Free Perfume Oil | 0.2 | 0.2 | 0.2 |
| Propellant ^{c} | 80 | 80 | 70 |

^{a} Alcohol 200 proof;
^{b} Tapioca Starch/Polymethylsilsesquioxane (Nouryon);
^{c} Isobutane/Propane (A-46)

TABLE 2
Dry Shampoo Compositions with Entrapped Perfume Oil

| | 4d | 4e | 4f |
|---|---|---|---|
| Example | [wt.-%] | [wt.-%] | [wt.-%] |
| Ethanol ^{a} | 0 | 5 | 20 |
| Dry Flo TS ^{b} | 5 | 5 | 5 |
| Isopropyl Myristate | 14.5 | 9.5 | 4.5 |
| Perfume Composition According to | 0.5 | 0.5 | 0.5 |
| Example 1 | | | |
| Propellant ^{c} | 80 | 80 | 70 |

^{a} Alcohol 200 proof;
^{b} Tapioca Starch/Polymethylsilsesquioxane (Nouryon);
^{c} Isobutane/Propane (A-46)

TABLE 3
Dry Shampoo Compositions with Entrapped Perfume Oil

| | 4g | 4h | 4i |
|---|---|---|---|
| Example | [wt.-%] | [wt.-%] | [wt.-%] |
| Ethanol ^{a} | 0 | 5 | 20 |
| Dry Flo TS ^{b} | 5 | 5 | 5 |
| Isopropyl Myristate | 14.5 | 9.5 | 4.5 |
| Perfume Composition According to | 0.5 | 0.5 | 0.5 |
| Example 3 | | | |
| Propellant ^{c} | 80 | 80 | 70 |

^{a} Alcohol 200 proof;
^{b} Tapioca Starch/Polymethylsilsesquioxane (Nouryon);
^{c} Isobutane/Propane (A-46)

### EXAMPLE 5: OLFACTIVE EVALUATION OF DRY SHAMPOOS

The olfactive evaluation was conducted on blotters (7.6 cm×12.7 cm).

The dry shampoo cans were shaken well and primed before testing, in order to ensure proper dispensing. For spraying the blotters the cans were held ca. 15 cm away from them. The cans were weighted before and after spraying each blotter to ensure uniformity. The average amount dispensed was 1.3 g.

After blotters were sprayed with the respective aerosol samples, also they were weighed ensure uniformity, and then placed on a bench top to dry. The average amount deposited was 0.2 g. The blotters were allowed to age for 8 h or 24 h, respectively.

Five untrained panelists were asked to smell the blotters prior to activation and give a score on a scale of 0-10, where 0 is the weakest and 10 being the strongest odor.

The panelists were then asked to activate the technology by holding the blotters with two hands, folding the blotter in half and rubbing the two-folded sides of the blotters back and forth 5 times. Again a score on a scale of 0-10, where 0 is the weakest and 10 being the strongest odor, was given.

The results are summarized in Table 4.

TABLE 4
Results of Olfactive Evaluation - 8 h of Aging

| | | Ethanol | | |
|---|---|---|---|---|
| | | Content | Pre-Activation | Post-Activation |
| Example | Perfume Format | [wt.-%] | Intensity | Intensity |
| 4a | Free Oil | 0 | 3.8 | 3.2 |
| 4b | | 5 | 4.0 | 2.2 |
| 4c | | 20 | 5.0 | 3.8 |
| 4d | Example 1 | 0 | 1.2 | 5.6 |
| 4e | | 5 | 3.4 | 5.8 |
| 4f | | 20 | 3.2 | 5.8 |
| 4g | Example 3 | 0 | 1.0 | 6.4 |
| 4h | | 5 | 3.4 | 5.0 |
| 4i | | 20 | 4.2 | 5.2 |

TABLE 5
Results of Olfactive Evaluation - 24 h of Aging

| | | Ethanol | | |
|---|---|---|---|---|
| | | Content | Pre-Activation | Post-Activation |
| Example | Perfume Format | [wt.-%] | Intensity | Intensity |
| 4a | Free Oil | 0 | 2.0 | 1.8 |
| 4b | | 5 | 2.4 | 2.4 |
| 4c | | 20 | 3.8 | 3.0 |
| 4d | Example 1 | 0 | 0.8 | 6.2 |
| 4e | | 5 | 2.2 | 6.0 |
| 4f | | 20 | 3.0 | 5.8 |
| 4g | Example 3 | 0 | 0.6 | 6.6 |
| 4h | | 5 | 2.0 | 5.4 |
| 4i | | 20 | 3.8 | 5.4 |

As apparent from Tables 4 and 5, dry shampoos containing free perfume oil (comparative examples 4a-4c) show no increased olfactive intensity upon friction activation, after 8 h and 24 h of aging. In contrast, products according to the present invention (examples 4e, 4f, 4 h and 4i) with perfume oil entrapped in a water-soluble matrix show good post activation intensity, after 8 h and 24 h of aging. A comparison between examples 4d-4f and 4g-4i, respectively, shows that this good friction activation is even achieved with an ethanol content as high as 20 wt.-%, relative to the total weight of the composition. This demonstrates that entrapment of the perfume oil in those compositions is surprisingly stable and resistant to leakage, even at such high ethanol concentrations. Furthermore, it is noted that the effect of friction activation is even more pronounced after 24 h than after 8 h. Also, there is a trend that perfume formulations according to Example 3 (Examples 4g-4i) perform better than perfume formulations according to Example 1 (Examples 4d-4f).

1. An anhydrous composition for treating a keratinous surface, the composition comprising:
at least one cosmetically acceptable excipient;
at least one perfume ingredient that is entrapped in a water-soluble matrix;
an alcohol;
wherein the amount of the alcohol in the composition is 1-30 wt.-%, relative to the total weight of the composition.

2. The composition according to claim 1, wherein the alcohol is selected from the group consisting of ethanol, isopropanol, ethylene glycol, propylene glycol, and glycerol, and mixtures thereof.

3. The composition according to one of claim 1, wherein the water-soluble matrix comprises at least one material selected from the group consisting of starch, maltodextrin, mannitol, chitosan, gum Arabic, alginate, cellulose, pectins, gelatin, polyvinyl alcohol and mixtures thereof.

4. The composition according to claim 17, wherein the water-soluble modified starch is selected from the group consisting of bleached starch, hydroxypropyl starch, hydroxypropyl distarch phosphate, dydroxypropyl distarch glycerol, acetylated distarch phosphate, starch acetate esterified with acetic anhydride, starch acetate esterified with vinyl acetate, acetylated distarch adipate, acetylated distarch glycerol, starch sodium octenyl succinate and mixtures thereof.

5. The composition according to claim 4, wherein the water-soluble matrix additionally comprises a compound selected from the group consisting of maltodextrin, mannitol and mixtures thereof.

6. The composition according to claim 4, wherein
the water-soluble matrix additionally comprises a hemicellulose.

7. The composition according to claim 1, wherein
the water-soluble matrix is in particulate form.

8. The composition according to claim 7, wherein the water-soluble matrix has a poured powder density of 300-600 g/l.

9. The composition according to claim 1, wherein
the composition has a water content of less than 5 wt.-%, relative to the total weight of the composition.

10. The composition according to claim 1, wherein
the at least one cosmetically acceptable excipient is selected from the group consisting of preservatives, antioxidants, chelating agents, sunscreen agents, vitamins, dyes, hair coloring agents, proteins, amino acids, plant extracts, humectants, oils, emollients, lubricants, butters, penetrants, thickeners, viscosity modifiers, polymers, resins, hair fixatives, film formers, surfactants, detergents, emulsifiers, opacifying agents, volatiles, liquid vehicles, carriers, salts, pH adjusting agents, neutralizing agents, buffers, hair conditioning agents, anti-static agents, anti-frizz agents, anti-dandruff agents, hair waving agents, hair straightening agents, relaxers, absorbents, deodorant agents, antiperspirant agents, and combinations thereof.

11. The composition according to claim 1, wherein the composition further comprises a propellant.

12. A consumer product comprising a composition according to claim 1.

13. The consumer product according to claim 12, wherein the consumer product is in the form of a spray.

14. The consumer product according to claim 13, wherein the consumer product is selected from the group consisting of dry shampoo sprays, hair sprays, styling sprays, deodorant sprays, antiperspirant sprays, cat litter refresher sprays, and combinations thereof.

15. A method for treating a keratinous surface,
the method comprising the step of applying to the keratinous surface a composition according to claim 1.

16. The method according to claim 15, wherein the method does not comprise a rinsing step.

17. The composition according to claim 3, wherein the starch is a water-soluble modified starch.

18. The composition according to claim 6, wherein the hemicellulose is a xyloglucan.

19. The method according to claim 15, wherein the keratinous surface is hair.

20. The method according to claim 19, wherein the hair is scalp hair.
